(19) United States
(12) Patent Application Publication
(10) Pub. No.: US 2016/0262793 A1
(43) Pub. Date: Sep. 15, 2016

(54) INSTRUMENT FOR PRODUCING A SKIN OPENING FOR MINIMALLY INVASIVE SURGERY

(71) Applicant: Gimmi GMBH, Tuttlingen (DE)

(72) Inventor: Josef Heinen, Tuttlingen (DE)

(21) Appl. No.: 14/643,794

(22) Filed: Mar. 10, 2015

(51) Int. Cl. A61B 17/34; A61B 17/3209

(52) U.S. Cl. CPC A61B 17/3417; A61B 17/32093; A61B 17/3496; A61B 2017/3454

(57) ABSTRACT

For producing a skin opening for minimally invasive surgery an instrument is employed, comprising a grip (10) and a puncture awl (16). The puncture awl (16) is provided on the distal end of a grip (10), which distal end forms a stop surface (12), which limits the penetration depth of the puncture awl (16).

### PRIORITY CLAIM

Priority is claimed of and to U.S. patent application Ser. No. 11/743,543, filed May 2, 2007, and German Application Serial No. 10 2006 020 595.2, filed on May 2, 2006, which are hereby incorporated herein by reference in their entirety.

### BACKGROUND OF THE INVENTION

1. Field of the Invention

The invention concerns an instrument for producing a skin opening for minimally invasive surgery, an instrument set for producing access for minimally invasive surgery, as well as a process for producing an access for minimally invasive surgery.

2. Background of the Invention

For a large number of surgical interventions the so-called minimally invasive surgery has become established. In this operation technique access to the field of operation lying within the body of the patient is obtained using a trocar sheath, through which surgical instruments, optical systems and the like can be introduced into the body. The advantage of this operation technique is comprised in particular therein, that the skin, and in certain cases thereunder lying tissue muscle layers, need only be opened a relatively small amount, so that only small operation wounds result. Thereby the pain associated with the operation wound is reduced, a more rapid healing is possible, and smaller operation scars result.

In order to be able to use the trocar sheath as an access way to the intracorporal field of operation, it is necessary to first produce an opening in the skin. For this, an incision is made using a scalpel, and the tissue below the skin in separated. This cut conventionally has a length of 10 to 15 mm, and represents an operation wound which must heal and can lead to scarring.

The invention is concerned with the task of further reducing the opening of the body surface for minimally invasive surgery.

### SUMMARY OF THE INVENTION

This task is inventively solved by an instrument, instrument set and by a process as described herein.

In accordance with the invention, the skin opening for the introduction of the trocar sheath is not produced by an incision using a scalpel. Rather, an instrument with a puncture awl or spur is used. The puncture awl is located distally on a grip, wherein the distal end of the grip, upon which the puncture awl is provided, forms a stop surface. The puncture awl has an axial length of approximately 5 to 10 mm. Distally the puncture awl terminates in a tip and widens in diameter towards its end bordering the stop surface to an approximate maximal diameter of 2 to 3 mm.

For opening the skin the puncture awl is stabbed into the skin, so that a hole is produced in the skin, of which the diameter corresponds to the maximal outer diameter of the puncture awl. The depth of the puncture hole is limited in that the stop surface of the grip contacts the skin surface and therewith a further penetration of the puncture awl is prevented. The maximal penetration depth is therewith determined by the axial length of the puncture awl. The depth of the produced puncture hole is actually greater than the axial length of the puncture awl, which is a consequence thereof, that the skin and the tissue layers lying under the skin are compressed by the axial pressure of the stop surface. The axial length of the puncture awl is approximately 5 to 10 mm, whereby a depth of the puncture hole of approximately 10 to 15 mm results. The puncture depth is sufficient to penetrate the puncture resistant epidermis and to provide an opening for the introduction of a trocar sheath and trocar or an obturator. A damaging of the vessels underlying the epidermis or organs underlying the skin is precluded by limiting of the penetration depth. The tissue layers lying under the epidermis offer less resistance, so that they can be penetrated by the sharp tipped trocar sheath or, as the case may be, by a blunt tipped obturator, if these are to be inserted into the epidermis through the penetration hole.

The diameter of the penetration hole produced using the penetration awl corresponds maximally to the maximal diameter of the penetration awl and is thus approximately 2 to 3 mm. The trocar sheath is introduced through this puncture hole, in which case a pointed trocar or a blunt obturator is situated for penetration of the tissue. The outer diameter of the trocar sheath is at least as large as the diameter of the puncture awl or, as the case may be, the skin opening produced thereby. Preferably the outer diameter of the trocar sheath is somewhat larger than the maximal diameter of the puncture awl, preferably approximately 0.5 mm larger, and maximally approximately 1 mm larger. Thereby the skin opening produced by the puncture awl is dilated by introduction of the trocar sheath, wherein the widening of the skin opening essentially occurs by atraumatic displacement of the skin tissue. As the trocar sheath is removed again after conclusion of the operation, the skin opening can draw together elastically to the diameter of the original puncture hole produced by the puncture awl.

The inventive operation technique thus requires only a minimal puncture as the access way to the intracorporal operation field, which represents a substantial improvement in comparison to the incision by means of a scalpel. The small puncture hole is easier to close, and often a water-tight wound adhesive is sufficient to close the puncture hole following the operation. The minimal lesion of the puncture hole results in a very rapid healing, in which case wound-healing problems are practically precluded. The small wound surface reduces the penetration of microbes by at least approximately 50% in comparison to conventional minimally invasive operation techniques. The small puncture hole reduces the pain of the wound to a minimum. Cosmetically unappealing scar formation is reduced to a minimum.

Additional features and advantages of the invention will be apparent from the detailed description which follows, taken in conjunction with the accompanying drawings, which together illustrate, by way of example, features of the invention.

### BRIEF DESCRIPTION OF THE DRAWINGS

The following drawings illustrate exemplary embodiments for carrying out the invention. Like reference numerals refer to like parts in different views or embodiments of the present invention in the drawings.

In the following the invention will be described in greater detail on the basis of an illustrative embodiment shown in the figures. There is shown in:

FIG. 1: an instrument according to the invention in side view, and

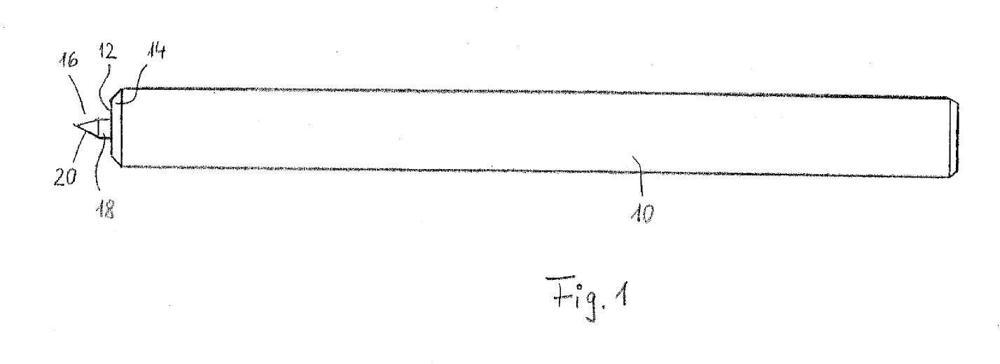

FIG. 2: an enlarged view of the distal end of the instrument.

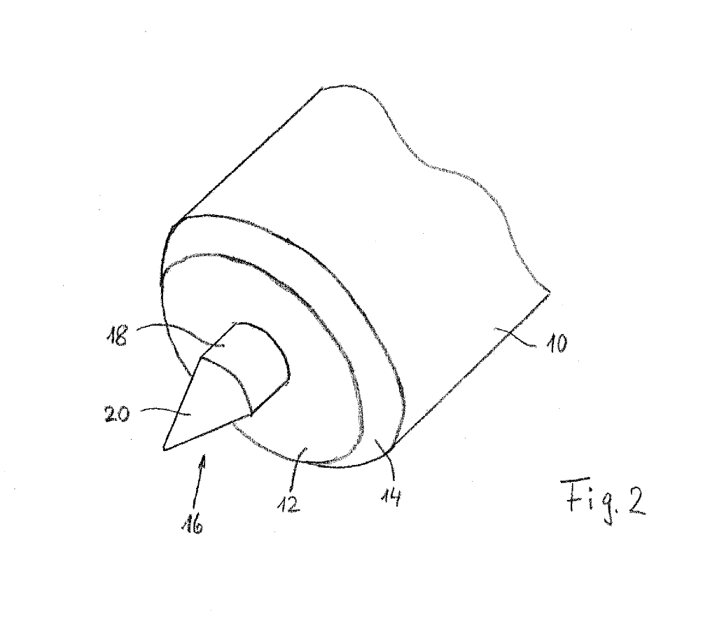

### DETAILED DESCRIPTION

Before the present invention is disclosed and described, it is to be understood that this invention is not limited to the particular structures, process steps, or materials disclosed herein, but is extended to equivalents thereof as would be recognized by those of ordinarily skilled in the relevant arts. It should also be understood that terminology employed herein is used for the purpose of describing particular embodiments only and is not intended to be limiting.

It must be noted that, as used in this specification and the appended claims, the singular forms “a” and “the” can include plural referents, unless the context clearly dictates otherwise. Thus, for example, reference to a “valve” can include reference to one or more of such valves.

### DEFINITIONS

In describing and claiming the present invention, the following terminology will be used in accordance with the definitions set forth below.

Relative directional terms, such as “distal,” “proximal,” “upper,” “lower,” “top,” bottom,” etc., are used herein to aid in describing various features of the present systems and techniques. It is to be understood that such terms are generally used in a manner consistent with the understanding one of ordinary skill in the art would have of such systems. Such terms should not, however, be construed to limit the present invention.

As used herein, the term “substantially” refers to the complete, or nearly complete, extent or degree of an action, characteristic, property, state, structure, item, or result. As an arbitrary example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained.

The use of “substantially” is equally applicable when used in a negative connotation to refer to the complete or near complete lack of an action, characteristic, property, state, structure, item, or result. As another arbitrary example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

Distances, forces, weights, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a numerical range of “about 1 inch to about 5 inches” should be interpreted to include not only the explicitly recited values of about 1 inch to about 5 inches, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value and should apply regardless of the breadth of the range or the characteristics being described.

Invention

The instrument includes a grip 10 which is elongated axially. In the illustrative embodiment the grip is similar to a pencil and has a generally cylindrical shape. The diameter of the grip 10 is so selected, that it is ergonomically comfortable to manipulate by hand. Accordingly, the grip 10 has a diameter of approximately 10 mm and an axial length of approximately 120 mm.

Of course the grip 10 can also have alternative shapes, in order to facilitate the manipulation of the instrument or to change the design. Accordingly, the cross section of the grip 10 need not be circular, but rather can be flattened, oval or polygonal. In the axial direction the grip 10 need not have constant diameter, but rather can have areas of narrowed and widened diameter. The surface of the grip 10 can be structured or textured, for example with circumscribing ridges, knurls, a roughening or the like.

The distal end surface of the grip 10 is in the form of a stop surface 12, which is perpendicular to the axis of the grip 10. The circumscribing edge of the contact surface 12 is reduced by a bevel 14.

Centrally in the stop surface 12, and therewith axially aligned with the center axis of the grip 10, a puncture awl 16 is provided, which projects axially in the distal direction and therewith perpendicular to the stop surface 12. The puncture awl 16 has an axial total length of approximately 5 to 8 mm. In its proximal area 18 adjacent the stop surface 12 the puncture awl is cylindrically shaped with a diameter of approximately 2 to 3 mm, preferably with a diameter of approximately 2.5 mm. In its distal area the puncture awl 16 transitions into a sharp tip 20, which narrows in the distal direction from the diameter of the proximal area 18. In the shown embodiment the tip 20 is conically shaped. It is of course possible that the tip 20 in its outer surface is bowed out spherically (convex) or shaped more tapered (concave). It is further possible that the tip 20 has a polygonal cross section, for example triangular or square. Finally, it is also possible that the cross section of the puncture awl 16 and in particular its proximal area 18 is not circular, but rather oval or polygonal.

The proximal area 18 and the tip 20 each respectively extend over approximately one half of the length of the puncture awl 16.

The grip 10 and the puncture awl 16 are preferably manufactured unitarily. As material, stainless steel is particularly preferred. Likewise, the instrument can be manufactured from plastic.

It is to be understood that the above-referenced arrangements are illustrative of the application for the principles of the present invention. Numerous modifications and alternative arrangements can be devised without departing from the spirit and scope of the present invention while the present invention has been shown in the drawings and described above in connection with the exemplary embodiments(s) of the invention. It will be apparent to those of ordinary skill in the art that numerous modifications can be made without departing from the principles and concepts of the invention as set forth in the examples.

1. An instrument for producing a skin opening for minimally invasive surgery, comprising: a grip; a stop surface defining a distal end of the grip; and a puncture awl provided on the grip within the stop surface and projects in a distal direction substantially perpendicularly; and wherein the puncture awl has an axial length of approximately 5 to 10 mm, distally terminates in a tip and in its proximal area adjacent the stop surface has a diameter of approximately 2 to 3 mm.

2. The instrument according to claim 1, wherein the puncture awl has a cylindrical area bordering the stop surface, which distally transitions into a tip.

3. The instrument according to claim 1, wherein the puncture awl has a substantially circular cross section.

4. The instrument according to claim 1, wherein the tip is approximately cone shaped (linear sides), or bowed out (convex) or bowed in (concave).

5. The instrument according to claim 2, wherein the proximal area and the tip respectively comprise one half of the puncture awl total axial length.

6. The instrument according to claim 1, wherein the grip extends axially and in which the puncture awl is provided axially parallel.

7. The instrument according to claim 6, wherein the grip is substantially shaped as a longitudinally extending cylinder having a distal end face that forms the stop surface.

8. The instrument according to claim 7, wherein the puncture awl is axially aligned with a center axis of the grip.

9. The instrument according to claim 1, wherein the grip and the puncture awl are unitary.

10. The instrument according to claim 9, wherein the grip and the puncture awl are formed of metal or plastic.

11. An instrument set for producing an access opening for minimally invasive surgery, comprising: instrument having a grip, a stop surface defining a distal end of the grip, and a puncture awl provided on the grip within the stop surface and projects in a distal direction substantially perpendicularly wherein the puncture awl has an axial length of approximately 5 to 10 mm, distally terminates in a tip and in its proximal area adjacent the stop surface has a diameter of approximately 2 to 3 mm; a trocar sheath; and a trocar and/or obturator adapted for passing through the trocar sheath, wherein an outer diameter of the trocar sheath is approximately 0 to 1.0 mm larger than a maximal diameter of the puncture awl.

12. A process for producing an access opening for minimally invasive surgery, comprising the steps of: piercing into skin with a puncture awl; producing a skin opening with a diameter of approximately 2 to 3 mm, in which the depth of penetration of the puncture awl is limited to approximately 10 mm by a stop surface; introducing a trocar or obturator into the skin opening via a trocar sheath wherein a distal sharp tip of the trocar or a dull tip of the obturator automatically dilates the puncture hole in diameter by up to 1 mm greater than the outer diameter of the trocar sheath; extracting the trocar or obturator from the trocar sheath; introducing operation instruments or optical systems through the trocar sheath into the intracorporal field of operation; and after ending the surgical intervention, extracting the trocar sheath from the skin opening, whereupon the skin opening elastically contracts and is reduced in diameter.

13. The process according to claim 12, wherein the skin opening diameter is approximately 2.5 mm.

14. The process according to claim 12, wherein puncture hole is dilated by approximately 0.5 mm greater than the outer diameter of the trocar sheath.
